(19) United States
(12) Patent Application Publication
(10) Pub. No.: US 2004/0148011 A1
(43) Pub. Date: Jul. 29, 2004

(54) Flexible connecting bar for an endoluminal stent graft

(76) Inventors: Brian Shiu, Sunnyvale, CA (US); Nickola Lewis, Santa Rose, FL (US); Ari Gershman, Cleaveland, OH (US); Feliks Basin, San Francisco, CA (US)

Correspondence Address: MEDTRONIC VASCULAR, INC., IP LEGAL DEPARTMENT, 3576 UNOCAL PLACE, SANTA ROSA, CA, 95403, US

(21) Appl. No.: 10/350,633

(22) Filed: Jan. 24, 2003

(51) Int. Cl. A61F 2/06

(52) U.S. Cl. 623/1.13

(57) ABSTRACT

A flexible connecting bar mounted on an endoluminal stent graft eliminates art problems associated with preferential bending. The flexible connecting bar has a length, and includes a plurality of articulation points along the length. The plurality of articulation points permits the flexible connecting bar to bend at least into and out of a plane.

### BACKGROUND OF THE INVENTION

1. Field of the Invention

The present invention relates generally to endoluminal stent grafts, and more particularly to structures for strengthening an endoluminal stent graft while minimizing the effects on flexibility of the endoluminal stent graft.

2. Description of Related Art

Various techniques have been used to enhance the flexibility of endoluminal stent grafts. For example, stents 101_1 . . . 101_7 (FIG. 1A) were spaced apart on The endoluminal graft material 105, which is typically a polyester, urethane, DACRON.RTM., TELON.RTM., or other suitable material, of endoluminal stent graft 100. Stents 101_1 . . . 101_7 were attached to graft material by polypropylene sutures (not shown), for example.

Gaps 102_1 . . . 102_6 between stents 101_1 . . . 101_7 allow more bending than with stents covering the entire graft material. However, the column strength of leg 110 becomes important.

To enhance the column strength of leg 110, a straight connecting bar 111 was sewn on leg 110 and attached to each of stents 101_3 . . . 101_7 through for example a crimped connection. While connecting bar 111 provided sufficient column strength, connecting bar 111 creates preferential bending direction of the stent graft with respect to the rotational position of the stent graft and the position of the connecting bar.

Specifically, when bending forces represented by arrows 121, 122 were applied so that connecting bar 111 and its side of the stent graft were under tension as in FIG. 1B, the connecting bar 111 bends as the graft material on the opposite side of the leg easily buckles to assume a smaller radius arc, as illustrated in FIG. 1C, and so did not impede the flexibility of leg 110. However, when bending forces represented by arrows 123, 124 were applied, the side of the stent graft opposite the connecting bar 111 would be pulled taut, and as the connecting bar 111 prevented buckling of its side of the stent graft and would not permit the side of the stent graft connected to the connecting bar from taking on a smaller radius arc, the stent graft effectively remained substantially stiff and straight as illustrated in FIG. 1D. Thus, the endoluminal stent graft with the straight connecting bar had a preferential bending characteristic.

### SUMMARY OF THE INVENTION

In one embodiment according to the present invention, a flexible connecting bar mounted on an endoluminal stent graft eliminates the prior art problems associated with preferential bending. The flexible connecting bar has a length, and includes a plurality of articulation points along the length. The plurality of articulation points permits the flexible connecting bar to bend at least into and out of a plane.

The plurality of articulation points is implemented in a variety of ways. In one embodiment of the articulation points, the plurality of articulation points includes coils. In another embodiment of the articulation points, the articulation points are defined by a periodic shape. For example, the periodic shape is a sinusoidal shape. In still another embodiment of the articulation points, at least one articulation point in the plurality of articulation points is defined by a wave shape. In still yet another embodiment of the articulation points, at least one articulation point in the plurality of articulation points is defined by a mushroom shape.

The flexible connecting bar is formed from Nitinol wire. The Nitinol wire is used in a martensitic phase of the Nitinol wire.

An endoluminal stent graft includes a leg and a plurality of stents spaced apart along the leg. A flexible connecting bar is attached to, i.e., mounted on, the leg.

One embodiment of the flexible connecting bar includes a plurality of coils. In another embodiment of the flexible connecting bar, the flexible connecting bar has a periodic shape such as a sinusoidal shape. In yet another embodiment of the flexible connecting bar, the flexible connecting bar includes a plurality of a wave shape. In still yet another embodiment of the flexible connecting bar, the flexible connecting bar includes a plurality of a mushroom shape. Alternatively, the flexible connecting bar includes a combination of the various shapes.

With the flexible connecting bar, the stents on the endoluminal stent graft are spaced apart and are used to provide radial strength for the endoluminal stent graft. At least one flexible connecting bar is attached to the endoluminal stent graft to provide column strength for the endoluminal stent graft. In one embodiment, a plurality of flexible connecting bars are attached to and spaced about the endoluminal stent graft to provide column strength for the endoluminal stent graft.

### BRIEF DESCRIPTION OF THE DRAWINGS

FIG. 1A is an illustration of a prior art endoluminal stent graft with spaced apart stents and a connecting bar with preferential bending characteristics.

FIG. 1B is an illustration of a portion of a leg of the prior art endoluminal stent graft of FIG. 1A under tension.

FIG. 1C is an illustration of the bending achieved from the tension of FIG. 1C.

FIG. 1D is an illustration of the portion of a leg of the prior art endoluminal stent graft of FIG. 1A under compression and illustrates that the leg remained relatively rigid and does not bend smoothly as in FIG. 1C.

FIG. 2A is an illustration of an endoluminal stent graft with spaced apart stents and a flexible connecting bar according to one embodiment of the present invention.

FIG. 2B is an illustration of the smooth bending achieved from the compression of FIG. 2A.

FIG. 2C is an illustration of the endoluminal stent graft of FIG. 2A under tension.

FIG. 2D is an illustration of the smooth bending achieved from the tension of FIG. 2C.

FIG. 3 is an illustration of an endoluminal stent graft with spaced apart stents and a flexible connecting bar where the flexible connecting bar has a periodic shape, such as a sinusoidal shape.

FIG. 4 is an illustration of an endoluminal stent graft with spaced apart stents and a flexible connecting bar where the flexible connecting bar includes a plurality of coils.

FIG. 5 is an illustration of an endoluminal stent graft with spaced apart stents and a flexible connecting bar where the flexible connecting bar includes a plurality of a wave shape.

FIG. 6 is an illustration of an endoluminal stent graft with spaced apart stents and a flexible connecting bar where the flexible connecting bar includes a plurality of a mushroom shape.

FIG. 7A is a top view of the flexible connecting bar of FIG. 3.

FIG. 7B is a side view of the flexible connecting bar of FIG. 3.

FIG. 8A is a top view of the flexible connecting bar of FIG. 4.

FIG. 8B is a side view of the flexible connecting bar of FIG. 4.

FIG. 9A is a top view of the flexible connecting bar of FIG. 5.

FIG. 9B is a side view of the flexible connecting bar of FIG. 5.

FIG. 10A is a top view of the flexible connecting bar of FIG. 6.

FIG. 10B is a side view of the flexible connecting bar of FIG. 6.

FIG. 11A is an illustration of a single flexible connecting bar attached to an endoluminal graph along the medial side of a leg.

FIG. 11B is an illustration of two flexible connecting bars attached in a first orientation about the leg of the endoluminal stent graft of FIG. 11A.

FIG. 11C is an illustration of two flexible connecting bars attached in a second orientation about the leg of the endoluminal stent graft of FIG. 11A.

FIG. 11D is an illustration of four flexible connecting bars attached in a third orientation about the leg of the endoluminal stent graft of FIG. 1A.

FIG. 12 is an illustration of the endoluminal stent graft of FIG. 3 with a plurality of flexible connecting bars mounted about the endoluminal stent graft.

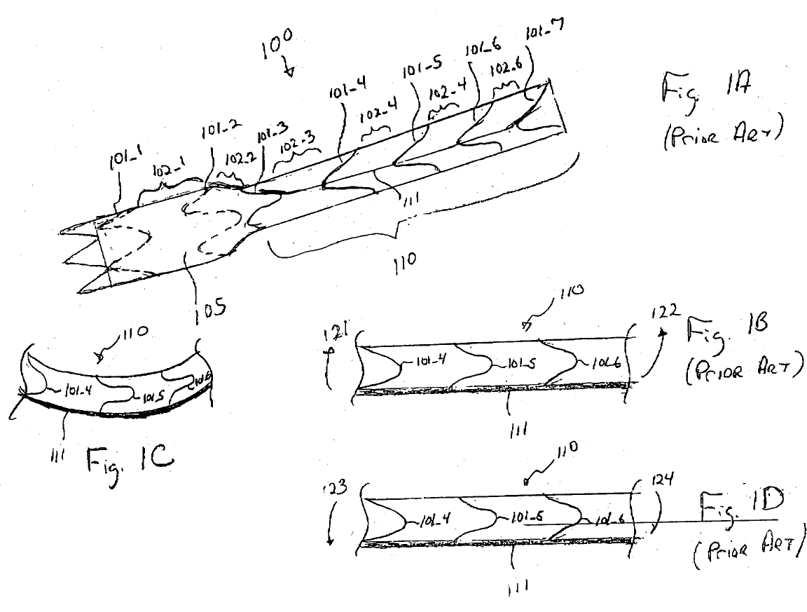

In the Figures and the following detailed description, elements with the same reference numeral are the same or equivalent elements.

### DETAILED DESCRIPTION

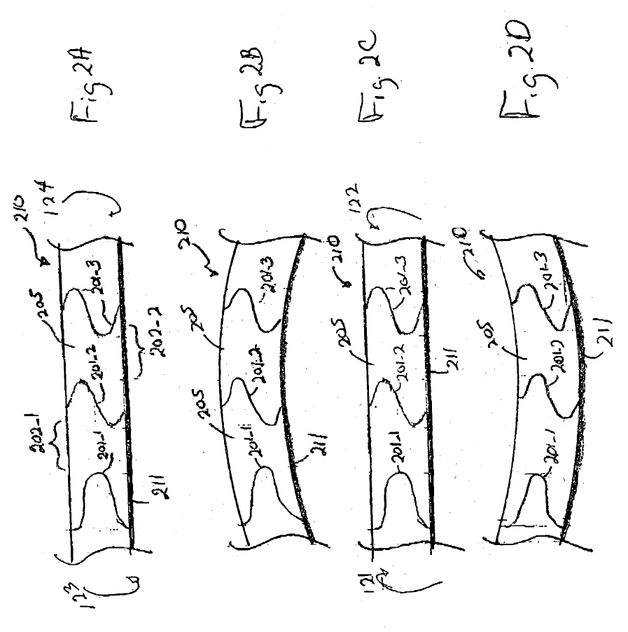

In one embodiment according to the present invention, a flexible connecting bar 211 (FIGS. 2A to 2D) is attached to an endoluminal stent graft 210 having a plurality of stents 201_1, 201_2, 201_3 spaced apart on a graft material 205. Unlike the prior art connecting bar whose flexibility was optimal only when configured on the outside of an arc in which the stent graft structure is being bent, i.e., in a preferred direction, flexible connecting bar 211 allows optimal bending in at least two directions, i.e., flexible connecting bar 211 accommodates both compression and extension while providing column strength. Hence, endoluminal stent graft 210 is more easily inserted and installed than the prior art endoluminal stent graft described above.

Specifically, when bending forces represented by arrows 123 and 124 are applied to flexible connecting bar 211, as illustrated in FIG. 2A, so that flexible connecting bar 211 is in compression, flexible connecting bar 211 bends smoothly as illustrated in FIG. 2B. Conversely when forces represented by arrows 121 and 122 are applied to flexible connecting bar 211, as illustrated in FIG. 2C, so that flexible connecting bar 211 is in tension, flexible connecting bar 211 again bends smoothly as illustrated in FIG. 2D.

Hence, in this embodiment, flexible connecting bar 211 bends in at least a first direction (FIG. 2B), and in a second direction opposite to the first direction (FIG. 2D). Since flexible connecting bar 211 bends in at least the two opposed directions, endoluminal stent graft 210 follows the configuration of a patient's lumen instead of forcing the patient's lumen to configure to the endoluminal stent graft.

In one embodiment, flexible connecting bar 211 achieves the multi-directional flexibility via a plurality of articulation points spaced along flexible connecting bar 211. These articulation points can be implemented in a number of different ways. Flexible connecting bar 211 is sewn on endoluminal stent graft 210 in one example.

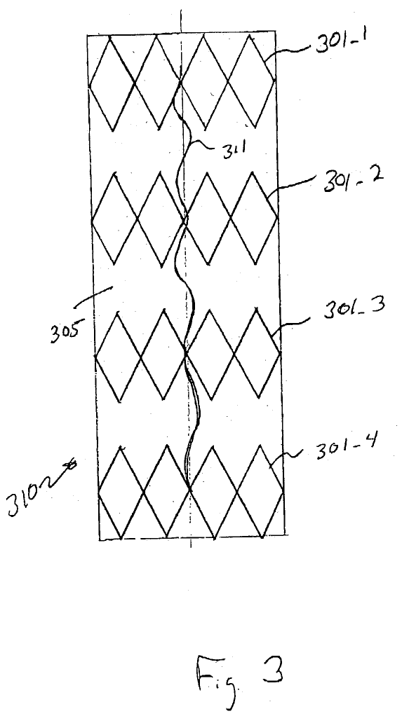

Flexible connecting bar 311 (FIG. 3) has a periodic shape, e.g., a sinusoidal wave shape and is attached to, i.e., mounted on, e.g., sewn on, an endoluminal stent graft 310 having a plurality of stents 301_1, 301_2, 301_3,301_4 that are spaced apart and mounted on a graft material 305. Each period of the periodic shape acts as at least one articulation point allowing flexible connecting bar to bend in a first direction and in a second direction opposite to the first direction.

In one embodiment, the periodic shape was formed in the connecting bar by using a mandrel with pins to shape the bar. The mandrel and shaped connecting bar was heat-treated in 505° C. salt for about five minutes.

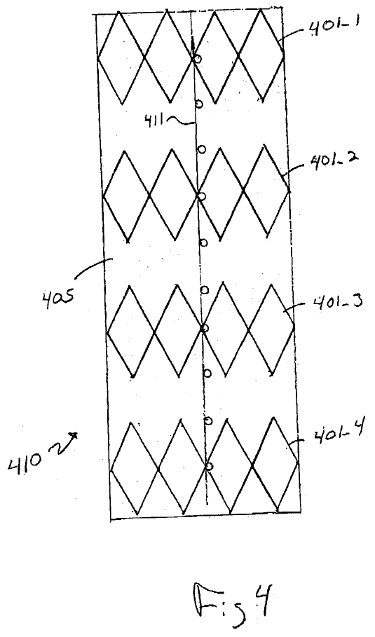

In another embodiment, flexible connecting bar 411 (FIG. 4) includes a plurality of coils, and is attached to, i.e., mounted on, an endoluminal stent graft 410 having a plurality of stents 401_1, 401_2, 401_3, 401_4 that are spaced apart and mounted on a graft material 405. The plurality of coils of flexible connecting bar 411 permits bending in a first direction as the coils. The plurality of coils also permits bends in a second direction opposite to the first direction with a coil acting as a pivot joint. Thus, the plurality of coils functions as the articulation points.

The plurality of coils of flexible connecting bar 411 was formed in connecting bar 411 by using a mandrel with pins to shape the bar, i.e., the wire was wrapped around a plurality of pins. The mandrel and shaped connecting bar were heat-treated in a salt bath at 505° C. for about five minutes.

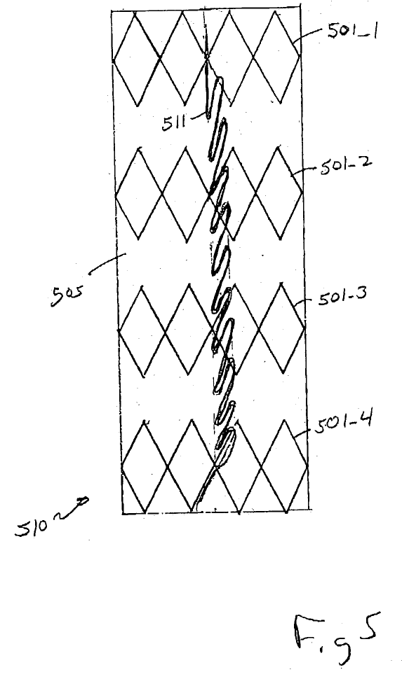

Flexible connecting bar 511 (FIG. 5) includes a plurality of a wave shape, and is attached, e.g., sewn, to an endoluminal stent graft 510 having a plurality of stents 501_1, 501_2, 501_3, 501_4 that are spaced apart and mounted on a graft material 505. The wave shape can be either periodic or non-periodic. Here, periodic means each wave shape has the same shape and size, or alternatively a set of wave shapes that can include waves of different shapes and sizes are repeated at uniform intervals. Again, the wave shape is formed from wire using a mandrel and pins and then heating treating the combination in a salt bath, as described above. Each wave shape functions as at least one articulation point.

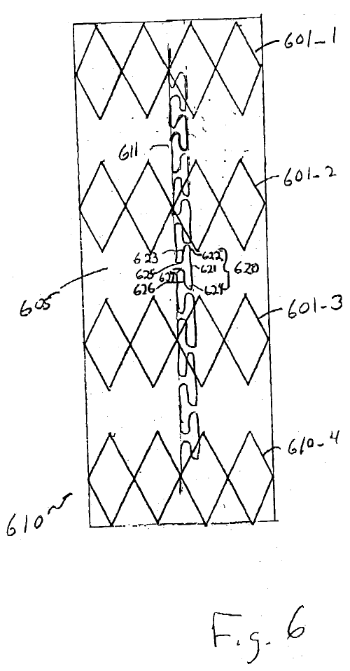

Flexible connecting bar 611 (FIG. 6) includes a plurality of a mushroom shape, and is attached to an endoluminal stent graft 610 having a plurality of stents 601_1, 601_2, 601_3, 601_4 that are spaced apart and mounted on a graft material 605. The mushroom shape can be either periodic or non-periodic. Here, periodic means each mushroom shape has the same shape and size, or alternatively a set of mushroom shapes that can include mushroom shapes of different shapes and sizes are repeated at uniform intervals. Again, this shape is formed from wire using a mandrel and pins and then the combination is heat treated in a salt bath, as described above. Each mushroom shape functions as at least one articulation point.

Mushroom shape 620 includes a flat top portion 621 that transitions into a first half-round portion 622 and a second half-round portion 624. First half-round portion 622 transitions into a first lower flat portion 623, while second half-round portion 624 transitions into a second lower flat portion 626. Flat top portion 621, first half-round portion 622, second half-round portion 624, first lower flat portion 623 and second lower flat portion 626 form the cap of the mushroom.

A third half-round portion 625 extends from first lower flat portion 623 while a fourth-half round portion 627 extends from second flat portion 626. Third half-round portion 625 and fourth half-round portion 627 form the stem of the mushroom. The description of half-round portions and flat portions are illustrative only and are not intended to limit the invention to these precise shapes. The actual shapes depend on the ability to bend the wire about the mandrels to form the mushroom shape.

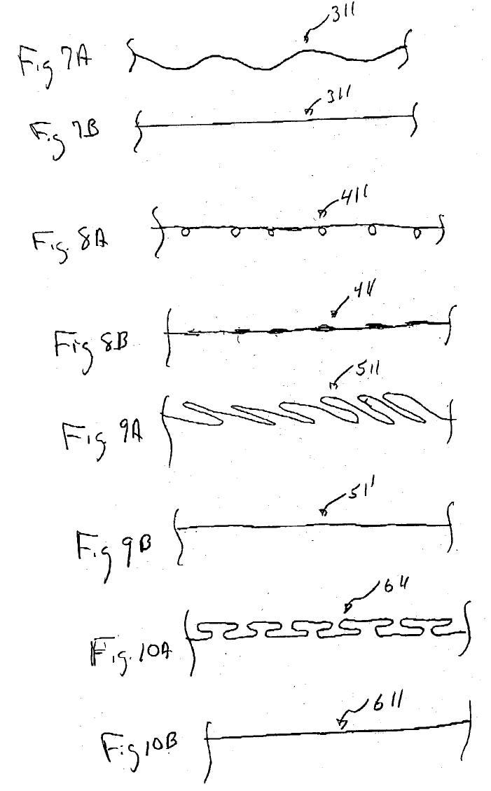

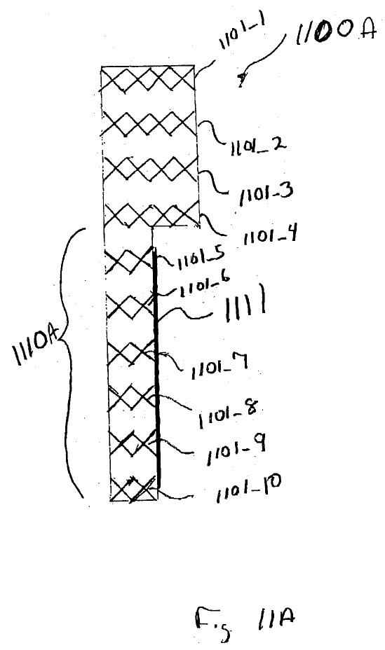

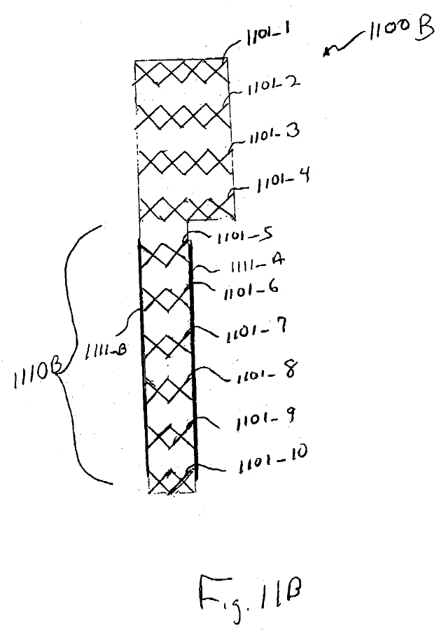

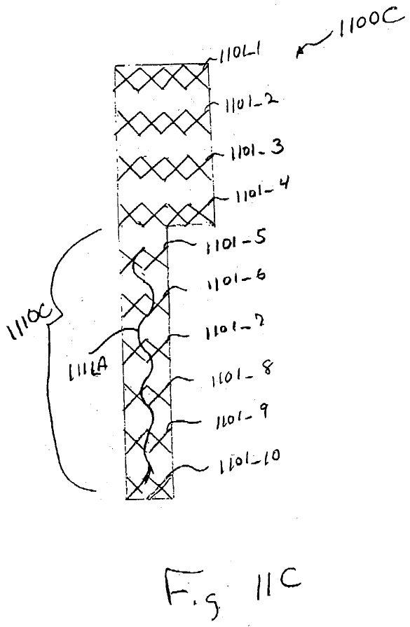

FIG. 7A is a top view of flexible connecting bar 311, while FIG. 7B is a side view of flexible connecting bar 311. FIG. 8A is a top view of flexible connecting bar 411, while FIG. 8B is a side view of flexible connecting bar 411. FIG. 9A is a top view of flexible connecting bar 511, while FIG. 9B is a side view of flexible connecting bar 511. FIG. 10A is a top view of flexible connecting bar 611, while FIG. 10B is a side view of flexible connecting bar 611.

As illustrated in FIGS. 7B, 9B, and 10B, the profile thickness of flexible connecting bars 311, 511, 611 is less than the profile thickness of flexible connecting bar 411. This is because the shapes of connecting bars 311, 511, and 611 are formed in one plane, while the coils of flexible connecting bar 411 extend out of the plane defined by the straight portions of flexible connecting bar 411.

With flexible connecting bars 311, 411, 511, and 611, the stents provide radial strength while flexible connecting bars 311, 411, 511, 611 provide column strength. Since the stents need only provide radial strength, in one embodiment, the stent geometry is selected to provide radial strength for the endoluminal graph, and a plurality of flexible connecting bars are used to provide column strength for the endoluminal graph. When a plurality of flexible connecting bars are used on the endoluminal stent graft, the flexible connecting bars are formed using a smaller diameter wire than when only a single flexible connecting bar is used.

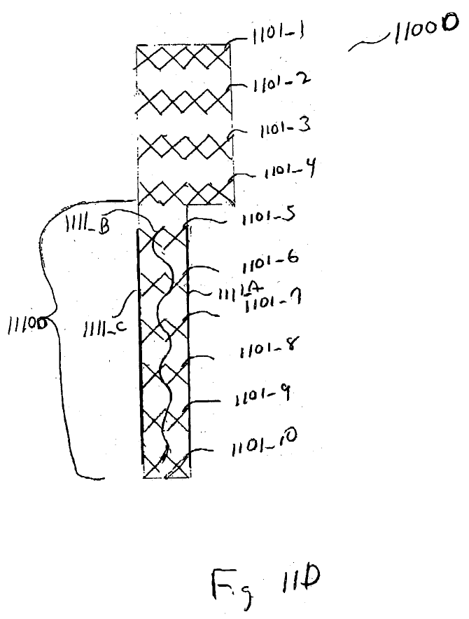

In FIG. 11A, a single flexible connecting bar 1111 is attached to an endoluminal stent graft 1100A along the medial side of a leg 1110A. In FIG. 11B, two flexible connecting bars 1111_A, 1111_B are attached to endoluminal stent graft 1100B in a first orientation about leg 1110B. In FIG. 11C, two flexible connecting bars 1111_A, 1111_B (not shown) are attached to endoluminal stent graft 1100C in a second orientation about leg 1110C. In FIG. 11D, four flexible connecting bars 1111_A, 1111_B, 1111_C, 1111_D (not shown) are attached to endoluminal stent graft 1100D in a third orientation. When using two or more flexible connecting bars, the diameters of the flexible connecting bars can be smaller than when using only a single flexible connecting bar.

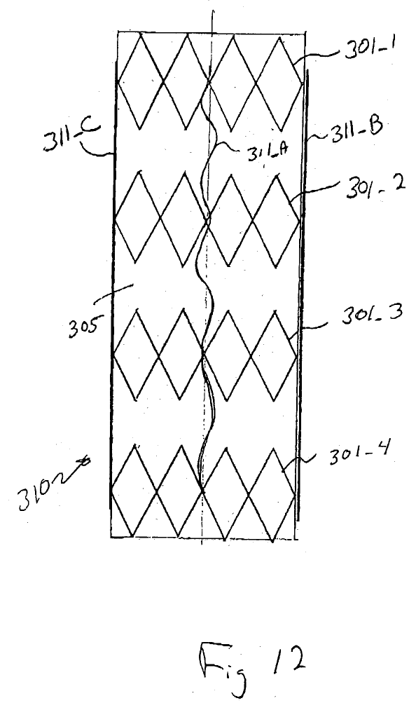

FIG. 12 is endoluminal stent graft 310 of FIG. 3 with a plurality of flexible connecting bars 311_A, 311B, 311C mounted on graft 310. In view of this disclosure, one of skill in the art can use a plurality of connecting bars to provide a desired column strength and flexibility. Hence, the combinations and orientations in FIGS. 11A to 11D and 12 are illustrative only and are not intended to limit the invention to the specific embodiments illustrated.

In one embodiment, the flexible connecting bar is a shape-memory metal in a martensitic phase of the shape-memory metal. For example, the flexible connecting bar is made from medical grade Nitinol wire. In the martensitic phase, the flexible connecting bar is softer and easier to flex and does not straighten out as it would in the superelastic austenitic phase. However, a flexible connecting bar in the austentic phase of the Nitinol wire could also be used.

In this embodiment, the flexible connecting bar was formed using pins and a mandrel, as described above, and then was heat treated to raise the austenite finish temperature Af to greater than room temperature. This simulated a martensitic condition at room temperature.

In one embodiment of FIG. 11A, flexible connecting bar 1111 is flexible connecting bar 311 formed of Nitinol wire in the martensitic phase. Flexible connecting bar 1111 is sewn onto a leg 1110 of endoluminal stent graft 1100 with a plurality of stents 1101_5 . . . 1101_10 with about a four to six millimeter spacing between stents. Flexible connecting bar 1111 is sewn along leg 1110 on the medial side.

In the various embodiments of this invention, the endoluminal graft material is a polyester, urethane, DACRON.RTM., TELON.RTM., or other suitable material. Also, in the various embodiments of the invention, the flexible connecting bar is from a medical grade material such as medical grade 316L stainless steel or other types of stainless steel, medical grade nickel-titanium alloys such a Nitinol, or medical grade nickel-titanium cobalt based alloys, or Nickel, Titanium & Cobalt based alloys (i.e., MP35 or 605L).

In one set of embodiments, the flexible connecting bar is formed from a wire having a diameter in a range from about 0.010-inch (0.025 cm) to about 0.020-inch (0.050 cm). In another set of embodiments, the wire diameter is in a range from about 0.010-inch (0.025 cm) to about 0.014-inch (0.036 cm). In one embodiment, the wire diameter is about 0.020-inch (0.050 cm). As described above, if a plurality of flexible connecting bars is used, the diameter of each wire can be smaller. In one embodiment, the wire size for a particular embodiment of an endoluminal stent graft is selected based upon empirical results including flexibility and column strength of the endoluminal stent graft. In another embodiment, the wire size and a number of attachment points to the endoluminal stent graft are selected based upon empirical results including flexibility and column strength of the endoluminal stent graft.

Thus, the embodiments described herein are only illustrative of flexible connecting bars with a plurality of articulation points for an endoluminal stent graft. In view of this disclosure, those of skill in the art make and use a flexible connecting bar that is appropriate for a particular graft. For example, a flexible connecting bar can use a combination of the shapes described herein or other shapes to obtain the desired multi-directional flexibility.

We claim:

1. An endoluminal stent graft comprising:
a flexible connecting bar having a length, and including a plurality of articulation points along said length.

2. The endoluminal stent graft of claim 1 wherein said articulation points comprise coils.

3. The endoluminal stent graft of claim 1 wherein at least one of said plurality of articulation points is defined by a periodic shape.

4. The endoluminal stent graft of claim 2 wherein said periodic shape comprises a sinusoidal shape.

5. The endoluminal stent graft of claim 1 wherein at least one of said plurality of articulation points is defined by a wave shape.

6. The endoluminal stent graft of claim 1 wherein at least one of said plurality of articulation points is defined by a mushroom shape.

7. The endoluminal stent graft of claim 1 wherein said flexible bar is formed from wire.

8. The endoluminal stent graft of claim 7 wherein said wire has a diameter in a range from about 0.010-inch to about 0.020-inch.

9. The endoluminal stent graft of claim 8 wherein said diameter is in a range from about 0.010-inch to about 0.014-inch.

9. The endoluminal stent graft of claim 8 wherein said diameter is about 0.020-inch.

10. The endoluminal stent graft of claim 1 wherein said flexible connecting bar is formed from Nitinol wire.

11. The endoluminal stent graft of claim 11 wherein said Nitinol wire is used in a martensitic phase of said Nitinol wire.

12. An endoluminal stent graft comprising:
a leg;
a plurality of stents spaced apart along said leg; and
a flexible connecting bar attached to said leg.

13. The endoluminal stent graft of claim 12 wherein said flexible connecting bar comprises a plurality of coils.

14. The endoluminal stent graft of claim 12 wherein said flexible connecting bar has a periodic shape.

15. The endoluminal stent graft of claim 14 wherein said periodic shape comprises a sinusoidal shape.

16. The endoluminal stent graft of claim 12 wherein said flexible connecting bar comprises a plurality of a wave shape.

17. The endoluminal stent graft of claim 12 wherein said flexible connecting bar comprises a plurality of a mushroom shape.

18. The endoluminal stent graft of claim 12 wherein said flexible connecting bar is formed from Nitinol wire.

19. The endoluminal stent graft of claim 18 wherein said Nitinol wire is used in a martensitic phase of said Nitinol wire

20. A method comprising:
using a plurality of spaced apart stents to provide radial strength for an endoluminal stent graft; and
using at least one flexible connecting bar to provide column strength for said endoluminal stent graft.

21. The method of claim 20 further comprising:
using a plurality of flexible connecting bars to provide column strength for said endoluminal stent graft, wherein said at least one flexible connecting bar is includes in said plurality of flexible connecting bars.

22. The method of claim 20 wherein said at least one flexible connecting bar is made from Nitinol wire and said Nitinol wire is in a martensitic phase.
